(19) United States
(12) Patent Application Publication
(10) Pub. No.: US 2025/0073437 A1
(43) Pub. Date: Mar. 6, 2025

(54) A COMBINED CATHETER-URETERAL STENT (CATH-STENT)

(71) Applicants: Sunil SINGH, Gurgaon (IN); Anil MANDHANI, Gurgaon (IN)

(72) Inventors: Sunil SINGH, Gurgaon (IN); Anil MANDHANI, Gurgaon (IN)

(21) Appl. No.: 18/718,068

(22) PCT Filed: Dec. 10, 2022
(86) PCT No.: PCT/IN2022/051064

§ 371 Date: Jun. 10, 2024

(30) Foreign Application Priority Data
Dec. 10, 2021 (IN) 202111025924

(51) Int. Cl. A61M 27/00; A61F 2/04

(52) U.S. Cl. CPC A61M 27/008; A61F 2/04; A61F 2002/047

(57) ABSTRACT

Disclosed is a catheter system (100). The catheter system (100) includes a stent (104) and a catheter (102). The catheter (102) includes a shaft (108) having a proximal end (114) and a distal end (116) such that the shaft (108) includes a first lumen (118) that extend from the proximal end (114) to the distal end (116) such that fluid is adapted to pass through the first lumen (118). The catheter (102) further includes a clamp (110) coupled to the distal end (116) of the shaft (108) such that upon passage of the fluid through the first lumen (118), the clamp (110) exhibits a closed position to hold the stent (104) and upon removal of the fluid from the first lumen (118), the clamp (110) exhibits an open position to release the stent (104).

### TECHNICAL FIELD

The present disclosure relates to a catheter, and more particularly relates to a combined catheter-ureteral stent device.

### BACKGROUND

Ureteral stents, also called double-J stents, are small silicone tubes placed inside the ureter of a subject for overcoming the obstruction to the flow of urine from kidneys due to stones or other causes and also after reconstructive surgery of the ureter. The ureteral stents are placed to relieve obstruction due to conditions such as, but not limited to, kidney stones, tumor, inflammation, ureteral kink or structure.

There are many situations when ureteric stent is required for a day to a week for which patients have to have an additional procedure for its removal. Some of the examples are ureteroscopy retrieval of stones leaving dust of the stone behind which could cause postoperative colic, live related transplant where stent is desired to be kept for a week, ureteroneocystostomy where double J stent is required for few days to overcome the problems of initial edema (tissue swelling) etc.

Conventionally, the ureteral stents are placed in the ureter of the subject by a tube supported on a guiding wire that is pushed from urethra via bladder and into the kidney. Once the first end of the tube is placed in the kidney with the other end in the bladder, the guide wire is withdrawn to make the stent curl at both the ends [hence the name double-J stents]. These curls help the tube or stent to be firmly held in across the ureter for longer time.

Foley catheter is a flexible tube placed inside the urethra for removing urine from urinary bladder of the subject. The tube has lumens for draining urine into a collection bag and for controlling a balloon for maintaining position of the catheter inside the urethra and bladder. While Foley catheter is taken out as it lies externally, ureteral stents are taken out by using cystoscopy, which is an invasive procedure and adds to the cost and discomfort to the patients.

Therefore, there exists a need of a device that can be used as a combination of catheter and the double J stent and should there be any need to leave the ureteral stent for longer time, then it should be possible to detach from the Foley catheter.

### SUMMARY

In view of the foregoing, a catheter system comprising: a stent and a catheter. The catheter includes a shaft having a proximal end and a distal end, such that the shaft includes a first lumen that extend from the proximal end to the distal end such that fluid is adapted to pass through the first lumen. The catheter further includes a clamp coupled to the distal end of the shaft such that upon passage of the fluid through the first lumen, the clamp exhibits a closed position to hold the stent and upon removal of the fluid from the first lumen, the clamp exhibits an open position to release the stent.

In some embodiments, the shaft further comprising second and third lumens such that the second and third lumens extend from the proximal end to the distal end of the shaft, wherein the fluid is adapted to pass through the second lumen.

In some embodiments, the catheter further comprising a balloon that is disposed at the distal end of the shaft such that upon passage of the fluid through the second lumen, the balloon exhibits an inflated position and upon removal of the fluid from the second lumen, the balloon exhibits a deflated position.

In some embodiments, the catheter system further comprising a guide-wire that is adapted to insert through the third lumen such that upon insertion of the guide-wire in the third lumen, the stent exhibits a straight position and upon removal of the guide-wire from the third lumen, the stent exhibits a coiled position.

In some embodiments, the first lumen further comprising first port that is disposed at the proximal end of the shaft such that the first port allows one of, ingress and egress of the fluid from the first lumen.

In some embodiments, the second lumen further comprising second port that is disposed at the proximal end of the shaft such that the second port allows one of, ingress and egress of the fluid from the second lumen.

In some embodiments, the catheter system is made up of a biocompatible material.

In some embodiments, the third lumen is configured to allow drainage of urine that is accumulated in a urinary bladder.

In some embodiments, the balloon when exhibits the inflated position, facilitates to maintain the position of the catheter within a urinary bladder.

In some embodiments, the balloon when exhibits the deflated position, facilitates to release the catheter from a urinary bladder.

### BRIEF DESCRIPTION OF DRAWINGS

The above and still further features and advantages of aspects of the present disclosure becomes apparent upon consideration of the following detailed description of aspects thereof, especially when taken in conjunction with the accompanying drawings, and wherein:

FIGS. 1A and 1B illustrates a front view of a catheter system, in accordance with an embodiment herein;

FIG. 2A illustrates a zoomed view of a clamp of a catheter in a closed position, in accordance with an embodiment herein;

FIG. 2B illustrates a zoomed view of a clamp of a catheter in an open position, in accordance with an embodiment herein; and

FIG. 3 illustrates a front view of the catheter system of FIG. 1 with a stent exhibiting a coiled position, in accordance with an embodiment herein.

To facilitate understanding, like reference numerals have been used, where possible, to designate like elements common to the figures.

### DETAILED DESCRIPTION

Various aspects of the present disclosure provide a catheter system. The following description provides specific details of certain aspects of the disclosure illustrated in the drawings to provide a thorough understanding of those aspects. It should be recognized, however, that the present disclosure can be reflected in additional aspects and the disclosure may be practiced without some of the details in the following description.

The various aspects including the example aspects are now described more fully with reference to the accompanying drawings, in which the various aspects of the disclosure are shown. The disclosure may, however, be embodied in different forms and should not be construed as limited to the aspects set forth herein. Rather, these aspects are provided so that this disclosure is thorough and complete, and fully conveys the scope of the disclosure to those skilled in the art. In the drawings, the sizes of components may be exaggerated for clarity.

It is understood that when an element or layer is referred to as being “on,” “connected to,” or “coupled to” another element or layer, it can be directly on, connected to, or coupled to the other element or layer or intervening elements or layers that may be present. As used herein, the term “and/or” includes any and all combinations of one or more of the associated listed items.

The subject matter of example aspects, as disclosed herein, is described with specificity to meet statutory requirements. However, the description itself is not intended to limit the scope of this disclosure. Rather, the inventor/inventors have contemplated that the claimed subject matter might also be embodied in other ways, to include different features or combinations of features similar to the ones described in this document, in conjunction with other technologies. Generally, the various aspects including the example aspects relate to a catheter system.

As already mentioned, there is a need to have a choice of removing double J stent which is put in to serve its purpose for short period of time. Accordingly, the catheter system 100 is provided herein.

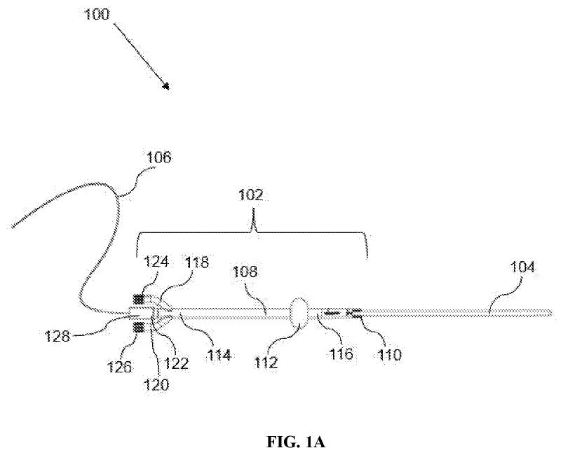

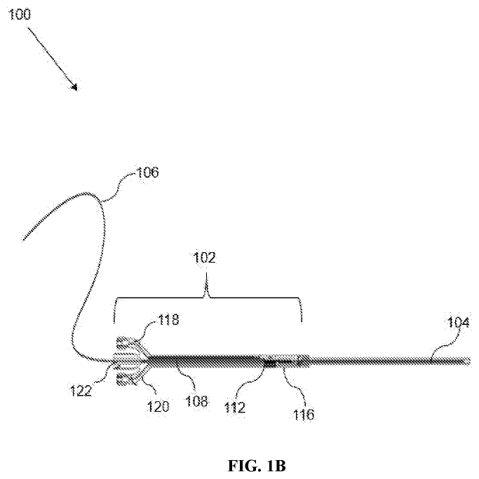

FIG. 1A and FIG. 1B illustrate a front view of a catheter system 100, in accordance with an embodiment herein. Specifically, FIG. 1A illustrates the front view of the catheter system 100 with inflated balloon, in accordance with an embodiment herein and FIG. 1B illustrates the front view of the catheter system 100 with deflated balloon, in accordance with an embodiment herein. The catheter system 100 may be inserted into a urinary bladder of a user to drain out the urine that is accumulated in the urinary bladder of the user. The term “user” as mentioned herein refers to a person who is using the catheter system 100. The catheter system 100 may be adapted to deploy a stent in a ureter of the user. The catheter system 100 may be used as Foley catheter.

In some embodiments, the catheter system 100 may be made up of a bio-compatible material.

The catheter system 100 may include a catheter 102, a stent 104, and a guide-wire 106. The catheter 102 may include a shaft 108, a clamp 110, and a balloon 112. The shaft 108 may include a proximal end 114, a distal end 116, and first through third lumens 118-122. The first through third lumens 118-122 may include first through third ports 124-128.

In some embodiments, the shaft 108 may be one of, a flexible, a tubular, and a hollow structure.

In some embodiments, the stent 104 may be a double-J stent.

The first through third lumens 118-122 may extend from the proximal end 114 to the distal end 116 of the shaft 108. The first through third ports 124-128 may be disposed at the proximal end 114 of the shaft 108. The first and second ports 124 and 126 may allow one of, ingress and egress of fluid from the first and second lumens 118 and 120 such that the fluid passes through the first and second lumens 118 and 120.

The balloon 112 may be disposed at the distal end 116 of the shaft 108. The balloon 112 may be adapted to exhibit an inflated position and a deflated position. Specifically, upon passage of the fluid through the second lumen 120, the balloon 112 may exhibit the inflated position and upon removal of the fluid from the second lumen 120, the balloon 112 may exhibit the deflated position. The balloon 112, when exhibits the inflated position, facilitates to maintain the position of the catheter 102 within the urinary bladder and the balloon 112, when exhibits the deflated position, facilitates to release the catheter 102 from the urinary bladder. Thus, the balloon 112 prevents slipping of the shaft 108 from the urinary bladder.

In some embodiments, a physician or an operator may control the inflated and deflated positions of the balloon 112 by way of controlling passage of the fluid through the second lumen 120. Specifically, the physician or the operator may pass the fluid through the second lumen 120 to facilitate the balloon 112 to exhibit the inflated position and the physician or the operator may remove the fluid from the second lumen 120 to facilitate the balloon 112 to exhibit the deflated position.

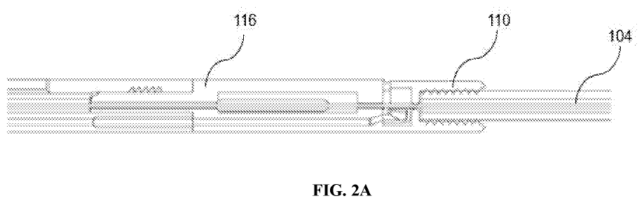

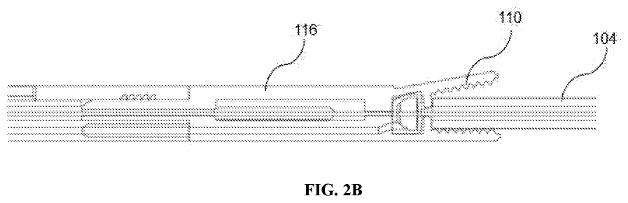

FIG. 2A illustrates a zoomed view of a clamp of a catheter in a closed position, in accordance with an embodiment herein. FIG. 2B illustrates a zoomed view of a clamp of a catheter in an open position, in accordance with an embodiment herein. The clamp 110 may be coupled to the distal end 116 of the shaft 108. The clamp 110 may be adapted to exhibit the closed position (as shown in FIG. 2A) and the open position (as shown in FIG. 2B). Specifically, upon passage of the fluid through the first lumen 118, the clamp 110 may exhibit the closed position to hold the stent 104 and upon removal of the fluid from the first lumen 118, the clamp 110 may exhibit the open position to release the stent 104 from the catheter 102 into the ureter of the user. The clamp 110, when exhibits the closed position, maintains the position of the stent 104 inside the ureter.

In some embodiments, the clamp 110 may prevent coiling of the stent 104 within the ureter of the user. In some embodiments, the clamp 110 may prevent slipping of the stent 104 within the ureter of the user.

In some embodiments, the physician may control the closed and open positions of the clamp 110 by way of controlling the passage of the fluid through the first lumen 118. Specifically, the physician may pass the fluid through the first lumen 118 to facilitate the clamp 110 to exhibit the closed position and the physician may remove the fluid from the first lumen 118 to facilitate the clamp 110 to exhibit the open position.

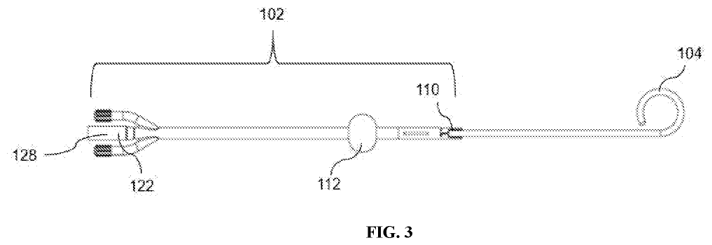

FIG. 3 illustrates a front view of the catheter system of FIG. 1 with a stent exhibiting a coiled position, in accordance with an embodiment herein. The third port 128 may facilitate insertion of the guide-wire 106 through the third lumen 122. The guide-wire 106 may facilitate the stent 104 to exhibit one of, a straight position and a coiled position. Specifically, upon insertion of the guide-wire 106 in the third lumen 122, the stent 104 may exhibit the straight position and upon removal of the guide-wire 106 from the third lumen 122, the stent 104 may exhibit the coiled position. The guide-wire 106 may facilitate correct positioning/coiling of the stent 104 inside the ureter or kidneys.

In some embodiments, the third lumen 122 may be configured to allow drainage of urine that is accumulated in the urinary bladder of the user such that the drained urine is collected in a collection bag.

In some embodiments, coiling of the stent 104, upon removal of the guide-wire 106 from the third lumen 122 may facilitate the upper end of the stent 104 to retain in the kidney.

In some embodiments, the physician may control the straight and coiled positions of the stent 104 by way of controlling the guide-wire 106. Specifically, the physician may insert the guide-wire 106 through the third lumen 122 to facilitate the stent 104 to exhibit the straight position and the physician may remove the guide-wire 106 from the third lumen 122 to facilitate the stent 104 to exhibit the coiled position.

In operation, the catheter system 100 may be inserted into the ureter of the user to drain the accumulated urine present inside the urinary bladder and to release the stent 104 inside the ureter. The guide-wire 106 may guide the stent 104 inside kidneys of the user. The guide-wire 106 may facilitate correct positioning of the stent 104 inside the kidneys. Specifically, upon insertion of the guide-wire 106 in the third lumen 122, the stent 104 may get straightened within the kidneys and upon removal of the guide-wire 106 from the third lumen 122, the stent 104 may get coiled within the kidneys. To deploy the stent 104 within the ureter, the guide-wire 106 may be removed from the third lumen 122. The coiling of the stent 104 may facilitate in broadening the ureter that allows the urine to flow from the kidneys to the urinary bladder through ureter.

While the balloon 112 reaches within the urinary bladder, the balloon 112 may be inflated by virtue of presence of the fluid inside the second lumen 120. The balloon 112, upon inflation, may be adapted to hold the catheter within the urinary bladder such that the urine that is accumulated inside the urinary bladder drains out through the third lumen 122.

Thus, the catheter system 100 may provide following advantages that may be derived from the structural and functional aspects of the catheter system 100: —
The catheter system 100 eliminates the need for removal of the stent using cystoscopy saving discomfort of an additional painful procedure and cost.

The foregoing discussion of the present disclosure has been presented for purposes of illustration and description. It is not intended to limit the present disclosure to the form or forms disclosed herein. In the foregoing Detailed Description, for example, various features of the present disclosure are grouped together in one or more aspects, configurations, or aspects for the purpose of streamlining the disclosure. The features of the aspects, configurations, or aspects may be combined in alternate aspects, configurations, or aspects other than those discussed above. This method of disclosure is not to be interpreted as reflecting an intention the present disclosure requires more features than are expressly recited in each claim. Rather, as the following claims reflect, inventive aspects lie in less than all features of a single foregoing disclosed aspect, configuration, or aspect. Thus, the following claims are hereby incorporated into this Detailed Description, with each claim standing on its own as a separate aspect of the present disclosure.

Moreover, though the description of the present disclosure has included description of one or more aspects, configurations, or aspects and certain variations and modifications, other variations, combinations, and modifications are within the scope of the present disclosure, e.g., as may be within the skill and knowledge of those in the art, after understanding the present disclosure. It is intended to obtain rights, which include alternative aspects, configurations, or aspects to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

We claim(s):

1. A catheter system (100) comprising:
a stent (104); and
a catheter (102) comprising:
a shaft (108) having a proximal end (114) and a distal end (116), wherein the shaft (108) comprising a first lumen (118) that extend from the proximal end (114) to the distal end (116) such that fluid is adapted to pass through the first lumen (118); and
a clamp (110) coupled to the distal end (116) of the shaft (108) such that upon passage of the fluid through the first lumen (118), the clamp (110) exhibits a closed position to hold the stent (104) and upon removal of the fluid from the first lumen (118), the clamp (110) exhibits an open position to release the stent (104).

2. The catheter system (100) as claimed in claim 1, wherein the shaft (108) further comprising second and third lumens (120 and 122) such that the second and third lumens (120 and 122) extend from the proximal end (114) to the distal end (116) of the shaft (108), wherein the fluid is adapted to pass through the second lumen (120).

3. The catheter system (100) as claimed in claim 2, wherein the catheter (102) further comprising a balloon (112) that is disposed at the distal end (116) of the shaft (108) such that upon passage of the fluid through the second lumen (120), the balloon (112) exhibits an inflated position and upon removal of the fluid from the second lumen (120), the balloon (112) exhibits a deflated position.

4. The catheter system (100) as claimed in claim 1 and 2, further comprising a guide-wire (106) that is adapted to insert through the third lumen (122) such that upon insertion of the guide-wire (106) in the third lumen (122), the stent (104) exhibits a straight position and upon removal of the guide-wire (106) from the third lumen (122), the stent (104) exhibits a coiled position.

5. The catheter system (100) as claimed in claim 1, wherein the first lumen (118) further comprising first port (124) that is disposed at the proximal end (114) of the shaft (108) such that the first port (124) allows one of, ingress and egress of the fluid from the first lumen (118).

6. The catheter system (100) as claimed in claim 2, wherein the second lumen (120) further comprising second port (126) that is disposed at the proximal end (114) of the shaft (108) such that the second port (126) allows one of, ingress and egress of the fluid from the second lumen (120).

7. The catheter system (100) as claimed in claim 1, wherein the catheter system (100) is made up of a biocompatible material.

8. The catheter system (100) as claimed in claim 2, wherein the third lumen (122) is configured to allow drainage of urine that is accumulated in a urinary bladder.

9. The catheter system (100) as claimed in claim 3, wherein the balloon (112) when exhibits the inflated position, facilitates to maintain the position of the catheter (102) within a urinary bladder.

10. The catheter system (100) as claimed in claim 3, wherein the balloon (112) when exhibits the deflated position, facilitates to release the catheter (102) from a urinary bladder.
